(19) United States
(12) Patent Application Publication
(10) Pub. No.: US 2026/0140682 A1
(43) Pub. Date: May 21, 2026

(54) DISPLAY DEVICE, DISPLAY CONTROL DEVICE AND DISPLAY SYSTEM

(71) Applicant: LG ELECTRONICS INC., Seoul (KR)

(72) Inventor: Gwanhui SEO, Seoul (KR)

(73) Assignee: LG ELECTRONICS INC., Seoul (KR)

(21) Appl. No.: 19/345,047

(22) Filed: Sep. 30, 2025

(30) Foreign Application Priority Data
Nov. 15, 2024 (KR) 10-2024-0163401

(51) Int. Cl. G06F 3/14

(52) U.S. Cl. CPC G06F 3/14

(57) ABSTRACT

There is disclosed a display control device configured to control an operation of a display device based on information related to a wearable device user, and an operation method thereof, and the display control device may include an input/output device communicatively connected to a wearable device and a display device; and at least one processor, and the at least one processor may be configured to obtain information related to a wearable device user transmitted by the wearable device; to determine an operation of the display device based on the obtained information related to the wearable device user; and to control the display device based on the determined operation.

This application claims priority to and the benefit of Korean Patent Application No. 10-2024-0163401, filed on November 15 2024, the disclosure of which is incorporated herein by reference in its entirety.

### TECHNICAL FIELD

Embodiments of the present disclosure relate to a display device, a display control device and a display system.

### BACKGROUND

As technology advances, the development of wearable devices is accelerating. A wearable device means a device that is wearable naturally on the body such as clothes, watches, glasses and accessories, and includes functions as a display device and a speaker.

Such wearable devices may perform data communication with various display devices go beyond simple display and provide various conveniences to wearable device users, going beyond simple display functions.

A smart TV refers to a multi-functional, intelligent and next-generation multimedia device configured to provide TV and Internet functions simultaneously by installing a web-based operation system OS on an existing TV.

Such a smart TV breaks away from one-sideness of existing broadcasting and provide a function that allows consumers to directly select the desired broadcast, and in addition to broadcasting content, the smart TV may also provide internet-based content, that are available on a PC or a smart phone such as internet web surfing, e-mail, games, photos and music and video media, to TV.

### SUMMARY

Accordingly, one object of embodiments of the present disclosure is to solve the above-noted disadvantages of the prior art, and embodiments of the present disclosure may provide a display device configured to control an operation of a display device based on information related to wearable device users, and a display control device.

Another object of the embodiments is to provide a display device that is configured to determine whether to operate a screen operation thereof based on preset information related to wearable device users, a display control device and a display system.

To solve the objects of the present disclosure, according to one embodiment, a display device may include an input/output interface communicatively connected to a wearable device and comprising a screen; and at least one processor. The at least one processor may be configured to obtain information related to a wearable device user transmitted by the wearable device, and to control an operation of the display device based on the obtained information related to the wearable device user.

According to another embodiment, a display control device may include an input/output device communicatively connected to a wearable device and a display device; and at least one processor. The at least one processor may be configured to obtain information related to a wearable device user transmitted by the wearable device; to determine an operation of the display device based on the obtained information related to the wearable device user; and to control the display device based on the determined operation.

According to a further embodiment, an operation method of a display device may include communicatively connecting to a wearable device; obtaining information related to the wearable device user transmitted by the wearable device; controlling an operation of the display device based on the obtained information related to the wearable device user.

According to a still further embodiment, an operation method of a display control device may include connecting to a wearable device, connecting to a display device, obtaining information related to a wearable device user transmitted by the wearable device, determining an operation of the display device based on the obtained information related to the wearable device user and controlling the display device based on the determining operation.

According to the embodiments of the present disclosure, the display device may have an effect of controlling at least one output operation of the display device based on preset information related to the wearable device user.

Furthermore, according to the embodiments of the present disclosure may have an effect of determining whether to operate the screen of the display device based on preset information related to the wearable device user.

### BRIEF DESCRIPTION OF DRAWINGS

FIG. 1 is a diagram showing the structure of a display device according to one embodiment of the present disclosure and connection with a wearable device and connection with a wearable device;

FIG. 2 is a diagram showing the structure of a display control device according to another embodiment of the present disclosure and connection with a wearable device and a display device;

FIG. 3 is a diagram showing an example in which information on wearable device users is displayed on a display device according to one embodiment;

FIG. 4 is a diagram showing an example in which wearable device user information is displayed on a display device according to one embodiment based on a valid distance;

FIG. 5 is a diagram showing the operations of a display control device and a display device according to another embodiment, when a wearable device user is sleeping;

FIG. 6 is a diagram showing the operations of a display control device and a display device based on activity information of a wearable device user according to one embodiment;

FIGS. 7A and 7B are diagrams showing operations of varying a user interface displayed on a screen of a display device based on a type of a display control device;

FIG. 8 is a flow chart showing an operation method of a display device according to one embodiment; and

FIG. 9 is a flow chart showing an operation method of a display control device according to another embodiment.

### DESCRIPTION OF EMBODIMENTS

The present disclosure may be variously modified and may have various embodiments, and particular embodiments illustrated in the drawings will be specifically described below.

The description of the embodiments is not intended to limit the present disclosure to the particular embodiments, but it should be interpreted that the present disclosure is to cover all modifications, equivalents and alternatives falling within the spirit and technical scope of the present disclosure.

It will be understood that the terms “first” and “second” are used herein to describe various components but these components should not be limited by these terms. The above terms are used only to distinguish one component from another. For example, a first component may be referred to as a second component and vice versa without departing from the scope of the invention. The singular expressions include plural expressions unless the context clearly dictates otherwise. Therefore, it is obvious that a first element mentioned below may also be a second element within the technical features of the present disclosure. Unless defined otherwise, all terms used herein, including technical or scientific terms, have the same meaning as commonly understood by those skilled in the art to which the present disclosure pertains. The terms defined in a generally used dictionary can be construed as meanings that match with the meanings of the terms from the context of the related technology and are not construed as an ideal or excessively formal meaning unless clearly defined in the present application.

It will be understood that when an element is referred to as being “connected with”, “on” or “coupled to” another element, the element can be directly connected with the other element or intervening elements may also be present. In contrast, when an element is referred to as being “directly connected with” another element, there are no intervening elements present. When describing relationship, for example, ‘between,’ ‘right between,’ ‘adjacent to,’ ‘directly adjacent to, etc. Or, unless ‘immediately’ or ‘directly’ is used, it should be understood the same as described above.

Throughout the disclosure, each component can be provided as a single one or a plurality of ones, unless explicitly stated to the contrary. A singular representation may include a plural representation unless it represents a definitely different meaning from the context. In understanding the components, it should be understood as including the error range even if there is no separate explicit description

Terms such as “include” or “has” been used herein and should be understood that they are intended to indicate an existence of several components, functions or steps, disclosed in the specification, and it is also understood that greater or fewer components, functions, or steps may likewise be utilized.

Terminology that is used in the present disclosure is limited to only for embodiments herewith but made only to make it easy to understand the present disclosure. Terms of respective elements used in the following description are terms defined taking into consideration of the functions obtained in the present invention.

Therefore, these terms do not limit technical elements in the present invention. Further, the defined terms of the respective elements will be called other terms in the art.

Below, preferred embodiments according to the disclosure are specifically described with reference to the accompanying drawings. In the drawings, identical reference numerals can denote identical or similar components. Description will now be given in detail according to exemplary embodiments disclosed herein, with reference to the accompanying drawings. For the sake of brief description with reference to the drawings, the same or equivalent components may be provided with the same reference numbers, and description thereof will not be repeated.

Hereinafter, exemplary embodiments will be described, referring to the accompanying drawings in detail.

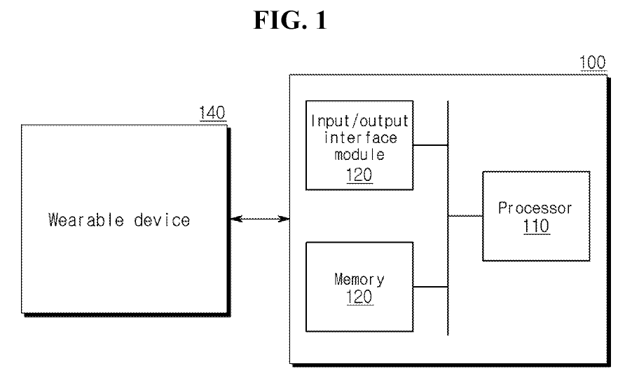

FIG. 1 is a diagram showing the structure of a display device according to one embodiment of the present disclosure and connection with a wearable device and connection with a wearable device.

Referring to FIG. 1, the display device 100 may be communicatively connected to a wearable device 140.

According to one embodiment, the display device 100 may be communicatively connected to the wearable device 140 using Bluetooth or Wi-Fi, but the communication method is not limited thereto.

Another device (e.g., a smart phone, etc.) may be used to connect the display device 100 and the wearable device 140 or to register the wearable device 140 to the display device 100.

Referring to FIG. 1, the display device 100 may include a processor 110, an input/output interface module 120 and a memory 130.

The processor 110, the input/output interface module 120 and the memory 130 that compose the display device 100 may be connected to each other and may transmit data to each other.

The processor 110 may be configured to execute programs or commands stored in the memory 130. At this time, an operating program (e.g., OS) for operating the display device 100 may be stored in the memory 130.

The processor 110 may be configured to execute a program for managing information about the display device 100.

The processor 110 may be configured to execute a program for managing the operation of the display device 100.

The processor 110 may be configured to execute a program for managing the operation of the input/output interface module 120.

The processor 110 may be configured to obtain information related to the wearable device user transmitted by the wearable device 140 through the input/output interface module 120. Here, the information related to the wearable device may be at least one of wearable device user biometric information, wearable device user location information and wearable device user motion information, but the information related to the wearable device user is not limited thereto. According to one embodiment, the information related to the wearable device user transmitted by the wearable device may be preset.

i) Biometric information display:

Information related to the wearable device user transmitted by the wearable device 140 may be biometric information of the wearable device user.

The processor 110 may obtain biometric information related to a wearable device user and may display it on a screen included in the input/output interface module 120. Here, biometric information may include body temperature information, heart rate information or sleep information.

The processor 110 may be configured to obtain biometric information related to the wearable device user transmitted by the wearable device 140 at a preset time interval (e.g., 10ms) through the input/output interface module 120. According to one embodiment, the wearable device 140 may transmit biometric information related to the wearable device user at a preset time interval, and the processor 110 may receive and obtain corresponding biometric information.

According to one embodiment, the processor 110 may receive the packet transmitted by the wearable device 140 and obtain the wearable device user’s biometric information.

The processor 110 may be configured to determine biometric information which will be displayed on a screen provided in the input/output interface module 120 among the obtained biometric information related to the wearable device user. Here, the biometric information determined to be displayed on the screen may include at least one of body temperature information, heat rate information or sleep information, but it is not limited thereto.

ii) Determining information display by determining presence of a valid distance:

The processor 110 may be configured to determine whether the wearable device user is within a valid distance from the display device 100 based on the number of packets received by the wearable device 140, and determine whether to display information related to the wearable device user based on the result of determination.

The processor 110 may be configured to count the number of packets transmitted by the wearable device 140. According to one embodiment, the processor 110 may count the number of packets received from the wearable device 140 for a preset time period (e.g., 1 second). For example, when the wearable device 140 transmits one packet every 10 ms, the processor 110 may receive 100 packets per second and the count value may be 100.

The processor 110 may be configured to determine whether to display information related to the wearable device user on a screen provided in the input/output interface module 120 based on the counted number of packets. If the counted number of packets is less than a preset number (e.g., 50), the processor 110 may determine not to display the information related to the wearable device user on the screen.

For example, when the wearable device user moves beyond the valid distance for normal communication between the wearable device 140 and the display device 100, the display device 100 may not normally receive packets transmitted by the wearable device 140, and the number of packets counted by the processor 110 may be less than a normal value (e.g., 100). In such cases, there may be no need to display the information about the wearable device user on the screen, because the wearable device user might be located far away from the screen of the display device 100.

If the processor 110 determines not to display the information related to the wearable device user on the screen provided in the input/output interface module 120, the processor 110 may not update the information related to the wearable device user at preset time intervals (e.g., 1 second).

iii) Turning on/off the display device based on determination of sleep state:

The processor 110 may determine whether the wearable device user is sleeping based on the information related to the wearable device user transmitted by the wearable device 140.

According to one embodiment, when heat rate information is included in the information related to the wearable device user transmitted by the wearable device 140, the processor 110 may determine whether the wearable device user is sleeping based on the heat rate information. For example, the processor 110 may determine that the wearable device user is sleeping, if the obtained heat rate information is less than or equal to a preset number of times.

According to another embodiment, if the information related to the wearable device user transmitted by the wearable device 140 includes information about whether the wearable device user is sleeping, the processor 110 may determine whether the wearable device user is sleeping based on the sleeping information.

The processor 110 may determine whether to operate the display device 100 based on the sleeping information about the wearable device user.

According to one embodiment, the processor 110 may determine whether to turn on/off the screen, to turn on/off the display device 100 and whether to activate/deactivate the always-on module based on the determination of the wearable device user’s sleeping.

When determining that the wearable device user is in a sleeping state, the processor 110 may turn off only the screen included in the input/output interface module 120, turn off the display device 100 or activate only the always-on module and turn off the other components of the display device 100.

According to one embodiment, if determining that the wearable device user is in a sleeping state, the processor 110 may turn off only the screen included in the input/output interface module 120, turn off the display device 100 or activate only the always-on module and turn off the other components of the display device 100 after a preset time (e.g., 10 minutes).

According to one embodiment, the always-on module may include some portion of the screen of the display device 100 (i.e., always-on screen). The processor 110 may display preset information on the always-on screen, when the always-on screen is activated. The preset information may include current time information, region information, current temperature information, wearable device user temperature information, heat rate information, sleeping state information, etc., but it is not limited to these. In this instance, the processor 110 may be continuously in communication with the wearable device 140 to periodically receive information related to the wearable device user from the wearable device 140 and update the related information displayed on the always-on screen.

ⅳ) Turning the display device on/off based on determination of the wearable device user’s activity state:

The processor 110 may determine a wearable device user’s activity state based on the information related to the wearable device user transmitted by the wearable device 140.

According to one embodiment, if heat rate information is provided in the information related to the wearable device user transmitted by the wearable device 140, the processor 110 may determine a wearable device user’s activity state based on the heat rate information. For example, if the obtained heat rate is a preset number of times or more, the processor 110 may determine that the wearable device user is in an active state.

According to another embodiment, if sleeping information is included in the information related to the wearable device user transmitted by the wearable device 140, the processor 110 may determine whether the wearable device user is in an active state (i.e., a non-sleeping state) based on the sleeping information.

The processor 110 may determine whether to operate the display device 100 based on the wearable device user’s activity state.

When determining that the wearable device user is in an active state, the processor 110 may determine whether to turn on the entire screen provided in the input/output interface module 120, turn on the display device 100 or determine to deactivate the always-on module.

ⅴ) Operation performing:

The processor 110 may determine whether to perform preset operations preset by the wearable device user based on the information related to the wearable device user transmitted by the wearable device 140.

According to one embodiment, the processor 110 may determine whether to output an alarm based on the obtained information related to the wearable device user. Here, the alarm type may be preset in the display device. According to one embodiment, the alarm may be a beep sound output from a speaker provided in the input/output interface module 120 or may display an over-the-air broadcast image on the screen, or may display a predesignated image on the screen, but the alarm type is not limited thereto.

According to one embodiment, when recognizing that a specific action (e.g., a double-pinch action) is performed by the wearable device user, the wearable device 140 may transmit trigger information related to this action to the display device 100.

When receiving the trigger information from the wearable device 140, the processor 110 may perform a preset operation related to the corresponding trigger information (e.g., an alarm).

According to one embodiment, the trigger information may include a plurality of trigger information tips. Actions for each trigger information data to be taken by the wearable device user and actions to be performed by the processor 110 may be preset. For example, when the wearable device is a ring worn on the finger of the wearable device user, one of various actions of the ring performed by the wearable device user may be set in response to each trigger information. For example, when recognizing that a circle is being drawn, the wearable device 140 may transmit first trigger information to the display device 100. If recognizing that a cross is being drawn, the wearable device 140 may transmit second trigger information to the display device 100. The processor 110 of the display device 100 may allow the display device 100 to perform an operation corresponding to the received first trigger information or second trigger information.

According to one embodiment, the processor 110 may recognize change in the state of the wearable device user such as a change from a sleep state to an active state, or a change from an active state to a sleep state, based on the information included in the packet periodically received from the wearable device 140. The processor 110 may recognize the change in the wearable device user state as a type of trigger information and preset an action corresponding to the trigger information. For example, when recognizing that the wearable device user is changing from an active state to a sleep state, the processor 110 may turn off the display device 100. Conversely, when recognizing that the wearable device user is changing from a sleep state to an active state, the processor 110 may turn on the display device 100.

When determining that the packet received from the wearable device contains trigger information by the wearable device user’s performing a preset action, the processor 110 may perform a preset action in response to the trigger information. The preset action may include displaying emergency status alarm information, displaying the wearable device user’s biometric information on the screen, increasing the volume, decreasing the volume, display home menu information on the screen, etc.

For example, when determining that it received the first trigger information, the processor 110 may output a preset alarm through a speaker provided in the input/output interface module 120. Here, the preset alarm may be a beep sound output from the speaker provided in the input/output interface module 120, but the preset alarm is not limited thereto.

ⅵ) Determining user interface:

The processor 110 may set a user interface displayed on the screen of the display device 100 differently based on the characteristics of the user using the display device 100 or the wearable device user. For example, the characteristics of the wearable device user may include age, and the processor 110 may be configured to display a user interface for seniors on the screen when the age of the wearable device user is greater than a preset value. The processor 110 may be configured to display a user interface for normal age on the screen when the age of the wearable device user is smaller than a preset value.

The processor may determine the type of user interface displayed on the screen of the display device 250 based on the type of the display device control device 200.

The processor 210 may transmit control information to the display device 250 so that the determined user interface type can be displayed on the screen of the display device 250.

The input/output interface module 120 may be communicatively connected to an external device (e.g., a wearable device, etc.) to transmit packets (or data) to the external device or receive packets (or data) from the external device. According to one embodiment, there may be at least one external device. The at least one external device may include a communication device such as a Bluetooth module, a Wi-Fi module, an infrared receiving module, etc.

The input/output interface module 120 may obtain a user’s input.

The input/output interface module 120 may include a user input device such as a keyboard and a mouse, to obtain the user’s input.

The input/output interface module 120 may include a screen, and video images and present information may be displayed on the screen.

The input/output interface module 120 may include a speaker, and may output voice/audio or preset alarm through the speaker.

The input/output interface module 120 may include a port (e.g., USB port) to be connected to an external device.

The input/output interface module 120 may include a monitor, a touchscreen, a mouse, an electronic pen, a microphone, a keyboard, a speaker, earphones, headphones or a touchpad.

The memory 130 may be configured to store various data and programs used in the display device 100.

The memory 130 may store the user input obtained by the input/output interface module 120, data received from the external device and data to be transmitted to the external device.

The memory 130 may store information configured to be displayed on a screen determined by the processor 110.

The memory 130 may store whether operate the screen determined by the processor 110.

The memory 130 may store whether to output the preset alarm determined by the processor 110.

The wearable device 140 may obtain at least one of biometric information, location information and activity information, which are related to the wearable device user.

The wearable device 140 may be communicatively connected to the display device 100 based on a preset method.

For example, the wearable device 140 may be connected to the display device 100 via Wi-Fi communication based on connection setting according to the Wi-Fi communication method. Alternatively, the wearable device 140 may be connected to the display device 100 via Bluetooth communication based on connection settings according to the Bluetooth communication method.

According to another embodiment, in order to quickly and simply establish the connection settings between the wearable device 140 and the display device 100, the wearable device 140 may be communicatively connected to the display device 100 by tagging the display device 100 (e.g., NFC tagging, etc.).

The wearable device 140 may obtain at least one of biometric information, location information and activity information of the wearable device user.

The wearable device 140 may transmit at least one of the obtained information related to the wearable device user to the connected display device 100.

Here, it is obvious to those skilled in the art that the term ‘device or module’ used herein indicates a logical configuration unit and not necessarily a physically distinct component.

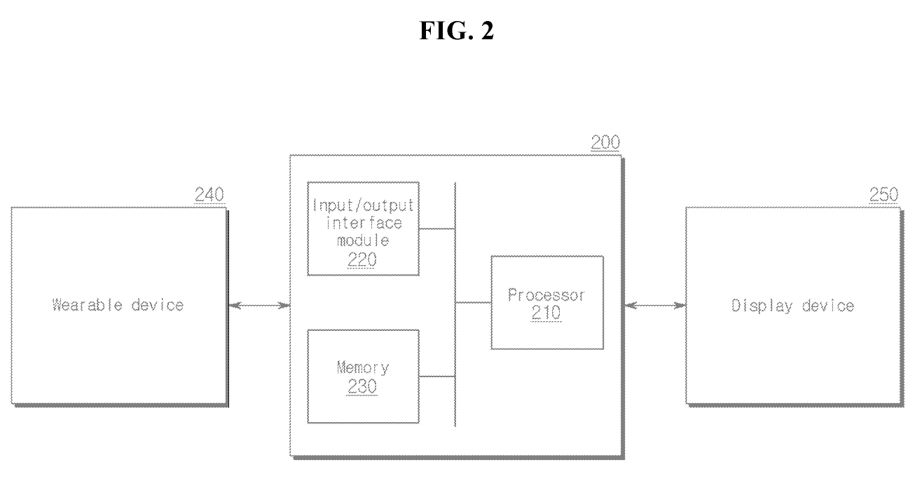

FIG. 2 is a diagram showing the structure of a display control device according to another embodiment of the present disclosure and connection with a wearable device and a display device.

Referring to FIG. 2, a display control device 200 may be separately provided from the display device 250 and may be communicatively connected to a wearable device 440 and a display device 250. At this time, the display device 250 may be normal television, and the display control device 250 may be provided with a function of a conventional remote controller configured to control the operation of the television using infrared rays.

According to one embodiment, the display control device 200 may recognize the wearable device 240 tagged based on a preset method (e.g., NFC tagging). The display control device 200 may transmit or receive setting data for Bluetooth or Wi-Fi communication through communication with a tagged wearable device 240, and may additionally connect Bluetooth or Wi-Fi communication.

Accordingly, the display control device 200 may be communicatively connected with the wearable device 140 via Bluetooth or Wi-Fi communication, but the communication method is not limited thereto.

Another device (e.g., a smart phone, etc.) may be used to connect the wearable device 240 and the display control device 200 or the wearable device 240 to the display control device 200.

The display control device 200 may be communicatively connected even to the display device 250 via Bluetooth communication.

The display control device 200 may simultaneously perform a Bluetooth master function and a Bluetooth slave function in order to be communicatively connected to both the display device 250 and the wearable device 240 through the Bluetooth method. According to one embodiment, the display control device 200 may operate as a Bluetooth master to communicate with the wearable device 240 and may operate as a Bluetooth slave to communicate with the display device 250.

Referring to FIG. 2, the display control device 200 may include a processor 210, an input/output interface module 220 and a memory 230.

The processor 210, the input/output interface module 220 and the memory 230, which constitute the display control device 200, may be interconnected and may transmit data to each other.

The processor 210 may be configured to execute the programs or commands stored in the memory 230. At this time, an operating program (e.g., OS) for operating the display control device 200 may be stored in the memory 230.

The processor 210 may be configured to execute a program for managing information about the display control device 200.

The processor 210 may be configured to execute a program for managing operations of the display control device 200.

The processor 210 may be configured to execute a program for managing operations of the input/output interface module 220.

The processor 210 may obtain information related to the wearable device user transmitted by the wearable device 240 through the input/output interface module 220. Here, the information related to the wearable device user may be at least one of biometric information, location information and activity information related to the wearable device user, but the information related to the wearable device user is not limited thereto. According to one embodiment, the information related to the wearable device user transmitted by the wearable device 240 may not be preset.

Hereinafter, the processor 210 of the display control device 200 may perform all the operations performed by the processor 110 of the display device 100 described above.

ⅰ) Displaying biometric information:

The information related to the wearable device user transmitted by the wearable device 240 may be biometric information about the wearable device user.

The processor 110 may obtain biometric information related to the wearable device user transmitted by the wearable device 140. Here, the biometric information may include temperature information, heat rate information or sleep information.

The processor 210 may be configured to obtain biometric information related to the wearable device user transmitted by the wearable device 240 at a preset time interval (e.g., 10ms) through the input/output interface module 220. According to one embodiment, the wearable device 240 may transmit biometric information related to the wearable device user at a preset time interval, and the processor 210 may receive and obtain corresponding biometric information.

According to one embodiment, the processor 210 may receive the packet transmitted by the wearable device 240 and obtain the wearable device user’s biometric information.

The processor 210 may be configured to determine biometric information which will be displayed on a screen provided in the display device 250 among the obtained biometric information related to the wearable device user. Here, the biometric information determined to be displayed on the screen may include at least one of body temperature information, heat rate information or sleep information, but it is not limited thereto.

The processor 210 may transmit the determined biometric information to the display device 250 through the input/output interface module 220.

According to one embodiment, the processor 210 may transmit the biometric information determined to be displayed on the screen of the display device 250 to the display device 250 through the input/output interface module 220 at a predetermined time interval (e.g., 1 second).

The processor 210 may transmit control information for controlling the display device 250 to display the biometric information on the screen, together with the biometric information determined to be displayed on the screen of the display device.

According to another embodiment, the processor 210 may set the display device 250 to display the received biometric information, when the display device 250 receives the biometric information transmitted by the processor 210. After that, the processor may periodically transmit only the biometric information determined to be displayed on the screen of the display device 250.

ii) Determining information display by determining presence of a valid distance:

The processor 210 may be configured to determine whether the wearable device user is within a valid distance from the display device 200 based on the number of packets received by the wearable device 240, and determine whether to display information related to the wearable device user based on the result of determination.

The processor 210 may be configured to count the number of packets transmitted by the wearable device 240. According to one embodiment, the processor 210 may count the number of packets received from the wearable device 240 for a preset time period (e.g., 1 second). For example, when the wearable device 240 transmits one packet every 10 ms, the processor 210 may receive 200 packets per second and the count value may be 100.

The processor 210 may be configured to determine whether to display information related to the wearable device user on the screen of the display device 250 based on the counted number of packets. If the counted number of packets is less than a preset number (e.g., 50), the processor 210 may determine not to display the information related to the wearable device user on the screen.

For example, when the wearable device user moves beyond the valid distance for normal communication between the wearable device 240 and the display device 200, the display device 200 may not normally receive packets transmitted by the wearable device 240, and the number of packets counted by the processor 210 may be less than a normal value (e.g., 100). In such cases, there may be no need to display the information about the wearable device user on the screen, because the wearable device user might be located far away from the screen of the display device 250.

If the processor 210 determines not to display the information related to the wearable device user on the screen of the display device 250, the processor 210 may stop transmitting the information related to the wearable device user to the display device 250 at preset time intervals (e.g., 1 second, etc.).

iii) Turning on/off the display device based on determination of sleep state:

The processor 210 may determine whether the wearable device user is sleeping based on the information related to the wearable device user transmitted by the wearable device 240.

According to one embodiment, when heat rate information is included in the information related to the wearable device user transmitted by the wearable device 240, the processor 210 may determine whether the wearable device user is sleeping based on the heat rate information. For example, the processor 210 may determine that the wearable device user is sleeping, if the obtained heat rate information is less than or equal to a preset number of times.

According to another embodiment, if the information related to the wearable device user transmitted by the wearable device 240 includes information about whether the wearable device user is sleeping, the processor 210 may determine whether the wearable device user is sleeping based on the sleeping information.

The processor 210 may determine whether to operate the display device 250 based on the sleeping information about the wearable device user.

According to one embodiment, the processor 110 may determine whether to turn on/off the screen, to turn on/off the display device 100 and whether to activate/deactivate the always-on module based on the determination of the wearable device user’s sleeping.

When determining that the wearable device user is in a sleeping state, the processor 210 may transmit control information such as turning off only the screen of the display device 250, turning off the display device 250 or activating only the always-on module and turning off the other components of the display device 250 to the display device 250. Accordingly, only the screen of the display device 250 may be turned off or the entire part of the display device may be turned off, or only the aways-on module of the display device may be activated. According to one embodiment, when transmitting only control information, the processor 210 may transmit the control information to the display device 250 by using a remote controller function, which is a traditional method of controlling the display device 250, without using Bluetooth communication that is communicatively connected to the display device 250.

According to one embodiment, if determining that the wearable device user is in a sleeping state, the processor 210 may turn off only the screen of the display device 250, turn off the display device 250 or activate only the always-on module and turn off the other components of the display device 250 after a preset time (e.g., 10 minutes).

According to one embodiment, the always-on module may include some portion of the screen of the display device 250 (i.e., always-on screen). The processor 210 may display preset information on the always-on screen, when the always-on screen is activated. The preset information may include current time information, region information, current temperature information, wearable device user temperature information, heat rate information, sleeping state information, etc., but it is not limited to these. In this instance, the processor 210 may be continuously in communication with the wearable device 240 to periodically receive information related to the wearable device user from the wearable device 240 and transmit information that should be displayed on the always-on screen to the display device 250.

ⅳ) Turning the display device on/off based on determination of the wearable device user’s activity state:

The processor 210 may determine a wearable device user’s activity state based on the information related to the wearable device user transmitted by the wearable device 240.

According to one embodiment, if heat rate information is provided in the information related to the wearable device user transmitted by the wearable device 240, the processor 210 may determine a wearable device user’s activity state based on the heat rate information. For example, if the obtained heat rate is a preset number of times or more, the processor 210 may determine that the wearable device user is in an active state.

According to another embodiment, if sleeping information is included in the information related to the wearable device user transmitted by the wearable device 140, the processor 110 may determine whether the wearable device user is in an active state (i.e., a non-sleeping state) based on the sleeping information.

The processor 110 may determine whether to operate the display device 100 based on the wearable device user’s activity state.

When determining that the wearable device user is in an active state, the processor 210 may determine whether to turn on the entire screen of the display device 250, turn on the display device 250 or determine to deactivate the always-on module. Then, the processor 210 may transmit subsequent control information to the display device 250. According to one embodiment, when transmitting only control information, the processor 210 may transmit the control information to the display device 250 by using a remote controller function, which is a traditional method of controlling the display device 250, without using Bluetooth communication that is communicatively connected to the display device 250.

ⅴ) Performing operation:

The processor 210 may determine whether to perform preset operations preset by the wearable device user based on the information related to the wearable device user transmitted by the wearable device 240.

According to one embodiment, the processor 210 may determine whether to output an alarm based on the obtained information related to the wearable device user. Here, the alarm type may be preset in the display device 250. According to one embodiment, the alarm may be a beep sound output from a speaker of the display device 250 may display an over-the-air broadcast image on the screen of the display device 250, or may display a predesignated image on the screen of the display device 250, but the alarm type is not limited thereto.

According to one embodiment, when recognizing that a specific action (e.g., a double-pinch action) is performed by the wearable device user, the wearable device 240 may transmit trigger information related to this action to the display device 250.

When receiving the trigger information from the wearable device 240, the processor 210 may transmit control information to the display device 250 so that the display device 250 may perform a preset operation related to the corresponding trigger information (e.g., an alarm).

According to one embodiment, the trigger information may include a plurality of trigger information tips. Actions for each trigger information data to be taken by the wearable device user and actions to be performed by the display device 250 may be preset. For example, when the wearable device is a ring worn on the finger of the wearable device user, one of various movements of the ring made by the wearable device user may be set in response to each trigger information. For example, when recognizing that a circle is being drawn, the wearable device 240 may transmit first trigger information to the display control device 200. If recognizing that a cross is being drawn, the wearable device 240 may transmit second trigger information to the display control device 200.

The processor 210 of the display device control device 200 may transmit control information allowing the display device 250 to perform an operation corresponding to the received first trigger information or second trigger information to the display device 250.

According to one embodiment, the processor 210 may recognize change in the state of the wearable device user such as a change from a sleep state to an active state, or a change from an active state to a sleep state, based on the information included in the packet periodically received from the wearable device 240. The processor 210 may recognize the change in the wearable device user state as a type of trigger information and preset an action corresponding to the trigger information. For example, when recognizing that the wearable device user is changing from an active state to a sleep state, the processor 210 may control the display device 250 to be turned off. Conversely, when recognizing that the wearable device user is changing from a sleep state to an active state, the processor 110 may control the display device 250 to be turned on.

When determining that the packet received from the wearable device contains trigger information by the wearable device user’s performing a preset action, the processor 210 may transmit a control signal for controlling the display device 250 to perform a preset action in response to the corresponding trigger information. The preset action may include displaying emergency status alarm information, displaying the wearable device user’s biometric information on the screen, increasing the volume, decreasing the volume, display home menu information on the screen, etc.

For example, when determining that it received the first trigger information, the processor 110 may transmit control information for controlling the display device 250 to output a preset alarm through a speaker to the display device 250. Here, the preset alarm may be a beep sound output from the speaker provided in the input/output interface module 120, but the preset alarm is not limited thereto.

ⅵ) Determining user interface:

According to one embodiment, the display control device 200 may be distinguished into a plurality of types. For example, the type of the display control device 200 may be a senior display control device or a conventional display control device, but the type of the display control device is not limited thereto. Such senior display control devices may have different exterior designs, such as larger buttons to make it easier for older people to press the buttons. The processor 210 of the display control device 200 may recognize whether the display control device is a senior display control device or a conventional display control device based on a hardware module such as a dip switch or a parameter value set by a soft ware module.

The processor 210 may determine the type of a user interface displayed on the screen of the display device 250 based on the type of the display control device 200. Here, the type of the user interface may be a user interface for seniors or a conventional user interface.

According to another embodiment, the display control device 200 may determine whether the wearable device user is a senior or not based on the information transmitted from the wearable device 240, and may determine the type of the user interface displayed on the screen of the display device 250 based on the determination result.

The processor 210 may transmit control information to the display device 250 so that the user interface determined based on the result may be displayed on the screen of the display device 250.

Referring to FIG. 2 again, the input/output interface module 220 may be communicatively connected to an external device (e.g., a wearable device, a display device, etc.) to transmit packets (or data) to the external device or receive packets (or data) from the external device. According to one embodiment, there may be at least one external device. So, the at least one external device may include a communication device such as a Bluetooth module, a Wi-Fi module, an infrared receiving module, etc. According to one embodiment, the input/output interface module 220 may include two Bluetooth modules, and one of the two may operate as a master and may be communicatively connected to the wearable device 240 operating as a slave. The other of the two may operate as a slave and may be communicatively connected to the display device 250 operating as a master.

The input/output interface module 220 may include a user input device such as a keyboard or a button configured to output a specific control signal, to obtain the user’s input.

The input/output interface module 120 may include a port (e.g., USB port) to be connected to an external device.

The memory 230 may be configured to store various data and programs used in the display control device 200.

The memory 230 may store the user input obtained by the input/output interface module 220, data received from the external device and data to be transmitted to the external device.

The memory 230 may store information configured to be displayed on the screen of the display device 250.

The memory 230 may store whether operate the screen determined by the processor 210.

The memory 230 may store whether to output the preset alarm determined by the processor 210.

The wearable device 240 may be communicatively connected to the display control device 200 based on a preset method.

The wearable device 240 may be tagged (e.g., NFC tagging) based on a preset method to be recognized by the display control device 200. Afterwards, through additional operations, the wearable device 240 may be connected to the display control device 200 via a second communication method such as Bluetooth or Wi-Fi.

The wearable device 240 may obtain at least one of biometric information about the wearable device user, location information about the wearable device user and activity information about the wearable device user.

The wearable device 240 may transmit the at least one obtained information to the display control device 200 among the biometric information, location information and activity information about the wearable device user.

The display device 250 may be communicatively connected to the display control device 200.

The display device 250 may be connected to the display control device 200 by using Bluetooth communication. In addition, the display device 250 may be controlled based on an infrared signal output from the display control device 200 in the same manner as being controlled by a conventional remote controller.

The display device 250 may display at least one of the biometric information, location information and sleep information about the wearable device user based on the received control signal or based on preset parameters.

The display device 250 may perform a preset operation corresponding to the received activity information of the wearable device user. According to one embodiment, the display device 250 may output a preset alarm based on the activity information of the wearable device user. According to one embodiment, the display device 250 may receive trigger information corresponding to the activity information of the wearable device user, and perform a preset operation in response to the corresponding trigger information.

The display device 250 may receive a type of a user interface to be displayed on the screen from the display control device 200, and display the corresponding type of the user interface on the screen. The display device 250 may receive the type of the user interface from the display control device 200 via Bluetooth communication or based on a remote controller function configured to emit an infrared including a control signal.

The display device 250 may display the determined user interface on the screen thereof.

Here, it is obvious to those skilled in the art that the term ‘device or module ‘ used herein indicates a logical configuration unit and not necessarily a physically distinct component

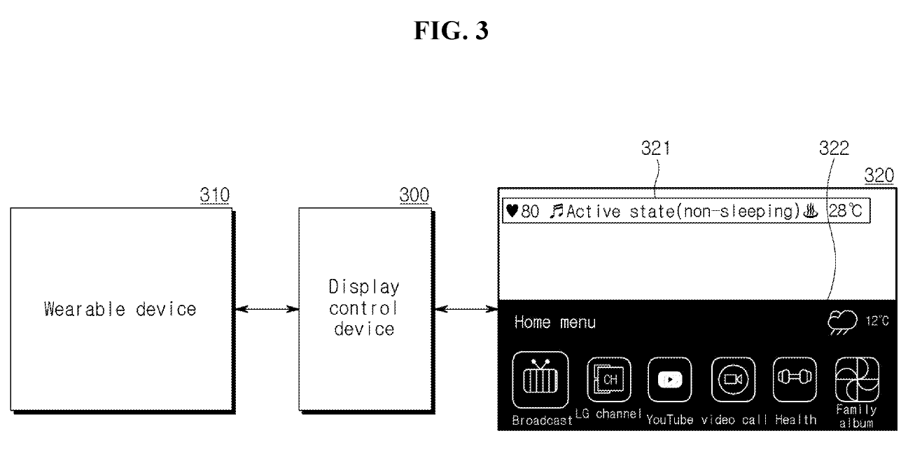

FIG. 3 is a diagram showing an example in which information on wearable device users is displayed on a display device according to one embodiment.

Referring to FIG. 3, the display device 300 may recognize a tagged wearable device 310 (e.g., NFC tagging, etc.) based on a preset method. here, the wearable device 310 may be a smart ring, a smart watch, etc., but the wearable device 310 is not limited thereto.

The display control device 300 may be communicatively connected to the wearable device 310 recognized by NFC tagging. According to one embodiment, the display device 300 may obtain data for communicative connection setting via NFC tagging.

Another device (e.g., a smart phone, etc.) may be used to connect the display control device 300 and the wearable device 310 or register the wearable device 310 to the display control device 300.

The display control device 300 may be connected to the display device 320 via Bluetooth communication. Here, the display device 320 may be a smart TV, but the display device 320 is not limited thereto.

According to one embodiment, the display control device 300 may function as a master to be connected to the wearable device 310, and function as a slave to be connected to the display device 320.

The wearable device 310 may obtain information related to the wearable device user. According to one embodiment, the wearable device 310 may obtain at least one of biometric information, location information and activity information about the wearable device user.

The wearable device 310 may transmit the obtained information related to the wearable device user to a connected display control device 300.

The wearable device 310 may transmit the information related to the wearable device user to the display control device 300 at a preset time interval (e.g., 10ms).

The display control device 300 may determine temperature information of the wearable device user, heat rate information of the wearable device user, or whether the wearable device user is sleeping, based on the information related to the wearable device user transmitted by the wearable device 310.

The display control device 300 may obtain the information related to the wearable device user transmitted by the wearable device 310 at a preset time interval (e.g., 10ms).

The display control device 300 may determine information that will be displayed on the screen of the display device 320 based on the information related to the wearable device user transmitted by the wearable device. According to one embodiment, the display control device 300 may determine to display heart rate information of the wearable device user, indoor temperature information, whether the wearable device user is sleeping 321, and home menu information 322 on the screen of the display device 320, but the information to be displayed on the screen of the display device 320 by the display control device 300 is not limited thereto.

The display control device 300 may transmit the information determined to be displayed on the display device 320 among the information related to the wearable device user transmitted by the wearable device 310 to the display device 320.

According to one embodiment, the display control device 300 may transmit the information determined to be displayed on the display device 320 to the display device 320 at a preset time interval (e.g., 1 second).

The display device 320 may receive a control signal and/or data transmitted by the display control device 300. According to one embodiment, the display device 320 may receive the data via Bluetooth communication, or the control signal via Bluetooth communication or infrared ray communication using a remote controller function.

The display device 320 may be operated based on the control signal received from the display control device 300.

The display device 320 may be configured to display biometric information 321 of the wearable device user received from the display control device 300 based on the control signal received from the display control device 300.

Furthermore, the display device 320 may display current temperature information. The display device 320 may receive the current temperature information from the display control device 300 or obtain it by measuring the current temperature.

Also, the display device 320 may display home menu information. The home menu information 322 may be preset and stored in the memory of the display device 320. According to one embodiment the home menu information 322 may be set differently based on the type of the user interface. For example, when receiving a control signal for displaying a user interface for seniors, the display device 320 may display home menu information for seniors on the screen. When receiving a control signal for displaying a user interface for general users, the display device 250 may display home menu information for normal users.

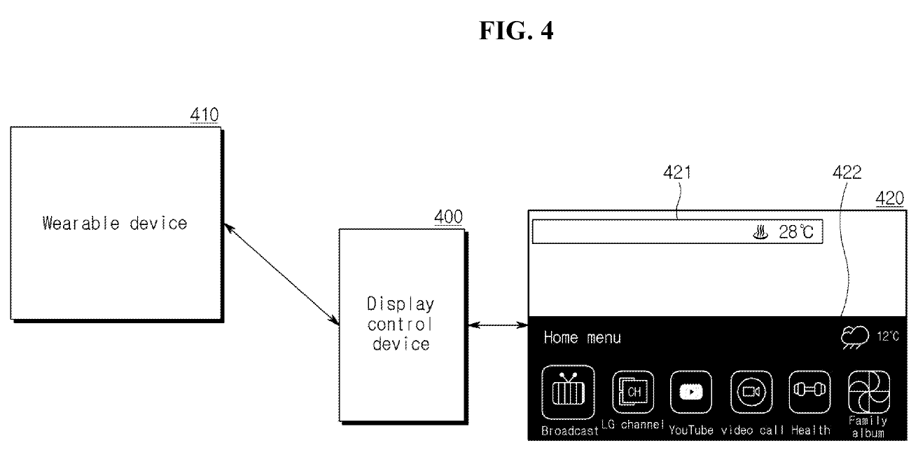

FIG. 4 is a diagram showing an example in which wearable device user information is displayed on a display device according to one embodiment based on a valid distance.

FIG. 4 shows the same connection configurations as FIG. 3, but the wearable device 410 may be located far away from the display control device 400.

Referring to FIG. 4, the display control device 400 may be connected to the display device 420 and the wearable device 410 shown in FIG. 4 in the same manner as described with respect to FIG. 3.

Referring to FIG. 4, the display control device 400 may recognize a wearable device 410 tagged by a preset method (e.g., NFC tagging).

The display control device 400 may be communicatively connected to the wearable device 410 recognized via the NFC tagging by using Bluetooth communication. According to one embodiment, the display control device 400 may obtain data for communication connection setting through NFC tagging.

The display control device 400 may be connected to the display device 420 by using Bluetooth communication.

The wearable device 410 may obtain information related to the wearable device user. The wearable device 410 may transmit the obtained information related to the wearable device user to the connected display control device 400. According to one embodiment, the wearable device 410 may transmit the information related to the wearable device user to the display control device 400 at a preset time interval (e.g., 10ms).

The display control device 400 may receive the packets transmitted by the wearable device 410 and obtain information related to the wearable device user at a preset time interval (e.g., 10ms)

The display control device 400 may be configured to count the number of packets transmitted by the wearable device 410. According to one embodiment, the display control device 400 may count the number of packets for a preset time period. For example, when the wearable device 410 transmits one packet every 10 ms, the display control device 400 may count 1000 packets for a preset time (e.g., 10 seconds).

Referring to FIG. 4, as moving after the wearable device 410 is connected to the display control device 400, the wearable device user may get away from the display control device 400. If the wearable device 410 gets farther away from the display control device 400, the wireless transmission environment deteriorates and the packets transmitted by the wearable device 410 may not be received by the display control device 400.

When the counted number of packets is less than a predetermined value (e.g., 500 packets for 10 seconds), the display control device 400 may determine that the wearable device 410 moves away beyond a valid distance and determined not to display the information related to the wearable device user on the screen of the display device 250.

When determining not to display the information related to the wearable device user on the screen of the display device 420, the display control device 400 may no longer transmit the information related to the wearable device user that was previously transmitted to the display device 420 at the preset time interval (e.g., 1 second).

Accordingly, as shown in FIG. 4, the display device 420 may no longer display the information related to the wearable device user that was displayed after received from the display control device 400, and may only home menu information 421 and temperature information.

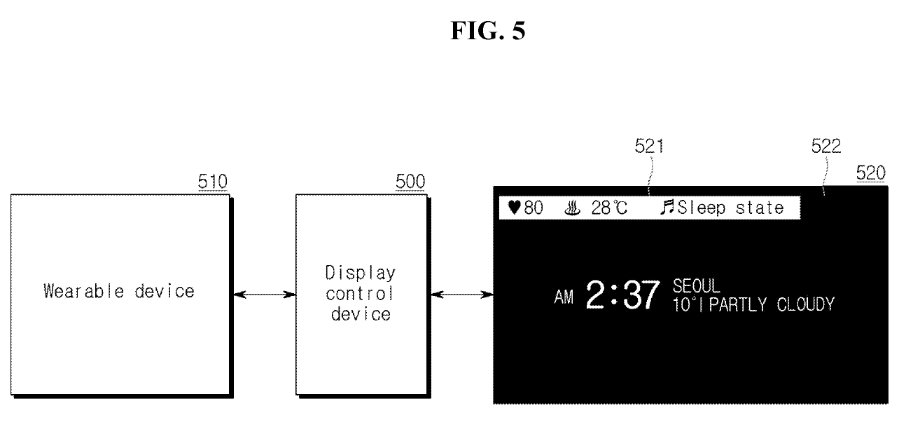

FIG. 5 is a diagram showing the operations of a display control device and a display device according to another embodiment, when a wearable device user is sleeping.

FIG. 5 shows the same connection configurations as FIG. 3, but the wearable device user is in a sleep state.

Referring to FIG. 5, the display control device 500 may be connected to the display device 520 and the wearable device 510 shown in FIG. 5 in the same manner as described with respect to FIG. 3.

Referring to FIG. 5, the display control device 500 may recognize a wearable device 510 tagged by a preset method (e.g., NFC tagging).

The display control device 500 may be communicatively connected to the wearable device 510 recognized via the NFC tagging by using Bluetooth communication. According to one embodiment, the display control device 500 may obtain data for communication connection setting through NFC tagging.

The display control device 500 may be connected to the display device 420 by using Bluetooth communication.

The wearable device 510 may obtain information related to the wearable device user. According to one embodiment, the wearable device 510 may obtain at least one of the biometric information, location information and activity information of the wearable device user.

The wearable device 510 may transmit the information related to the wearable device user to the connected display control device 500. According to one embodiment, the wearable device 510 may transmit the information related to the wearable device user to the display control device 500 at a preset time interval (e.g., 10ms).

The display control device 500 may obtain the information related to the wearable device user by receiving the packets transmitted by the wearable device 510 for a preset time interval (e.g., 10ms). For example, the display control device 500 may obtain heat rate information of the wearable device user or information about whether the wearable device user is sleeping.

The display control device 500 may determine whether the wearable device user is sleeping based on the information related to the wearable device user transmitted by the wearable device 510.

According to one embodiment, when heat rate information is included in the information related to the wearable device user transmitted by the wearable device 510, the display control device 500 may determine whether the wearable device user is sleeping based on the heat rate information. For example, the display control device 500 may determine that the wearable device user is sleeping, if the obtained heat rate information is less than or equal to a preset number of times.

According to another embodiment, if sleeping information is included in the information related to the wearable device user transmitted by the wearable device 510, the display control device 500 may determine whether the wearable device user is sleeping based on the sleeping information.

The display control device 500 may determine whether to operate the display device 520 based on whether the wearable device user is sleeping. Here, the operation of the display device 520 may be turning the screen of the display device 520 on/off, turning the display device 500 on/off and activating/deactivating the always-on of the screen of the display device 520. However, the operation of the display device 520 is not limited thereto.

According to one embodiment, when determining that the wearable device user is in a sleep state, the display control device 500 may continuously monitor whether the wearable device user is sleeping for a preset time (e.g., 10 minutes). When determining that the wearable device user is continuously sleeping, the display control device 500 may control the display device 520 to display the determined screen.

Referring to FIG, 5, according to one embodiment, the display control device 500 may control the display device 520 to activate the always-on display of the display device 520.

When the always-on display of the display device is activated, the display control device 500 may control the display device 520 to display the information related to the wearable device user. Here, the information related to the wearable device user may be heat rate information of the wearable device user or whether the wearable device user is sleeping 521. In addition, the display control device 500 may control current time information, regional information 522, and current temperature information to be further displayed on the always-on display screen of the display device 520.

In one embodiment shown in FIG. 5, the entire screen of the display device 520 is the always-on display screen. However, in another embodiment, a predetermined portion of the screen of the display device 520 may be the always-on screen.

According to this embodiment, when determining that the wearable device user is in an active state (i.e., a non-sleeping state), the display control device 500 may display a screen of the display device 320 shown in FIG. 3.

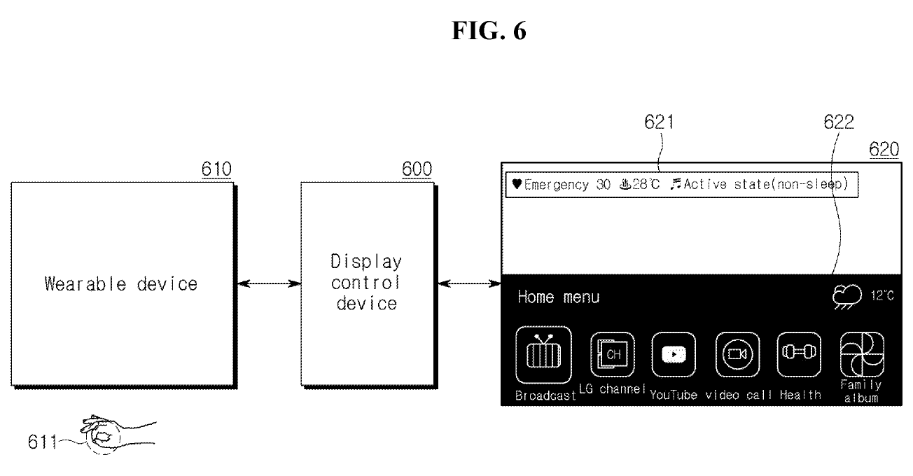

FIG. 6 is a diagram showing the operations of a display control device and a display device based on activity information of a wearable device user according to one embodiment.

FIG. 6 shows the same connection configurations as FIG. 3, but the wearable device user may perform a preset specific action.

Referring to FIG. 6, the display control device 600 may be connected to a display device 620 and a wearable device 610 shown in FIG. 6 in the same manner as described with respect to FIG. 3.

Referring to FIG. 6, the display control device 600 may recognize the wearable device 610 tagged by a preset method (e.g., NFC tagging).

The display control device 600 may be communicatively connected to the wearable device 610 recognized via the NFC tagging by using Bluetooth communication. According to one embodiment, the display control device 600 may obtain data for communication connection setting through NFC tagging.

The display control device 600 may be connected to the display device 420 by using Bluetooth communication.

The wearable device 610 may obtain information related to the wearable device user. According to one embodiment, the wearable device 510 may recognize that a specific action (e.g., a double-pinch action) is performed by the wearable device user. When recognizing the wearable device user performs a specific action, the wearable device 240 may transmit trigger information related to this action to the display control device 600. According to one embodiment the wearable device 610 may transmit trigger information to the display control device 600 at a preset time interval (e.g., 10ms).

The display control device 600 may transmit control information to the display device 620 so that the wearable device 610 can perform an operation set in relation to the trigger information transmitted by the wearable device 610. According to one embodiment, the display control device 600 may control the display device 620 to output an alarm preset for the trigger information received from the wearable device 610. Here, the preset alarm may be a beep sound output from the speaker of the display device 620 or “Emergency” displayed on the screen of the display device 620 shown in FIG. 6, or may output a beep sound and display “Emergency” simultaneously. However, the present alarm is not limited thereto.

According to another embodiment, the operation of the display device 620 set in relation to trigger information may include turning the display device 620 on or off.

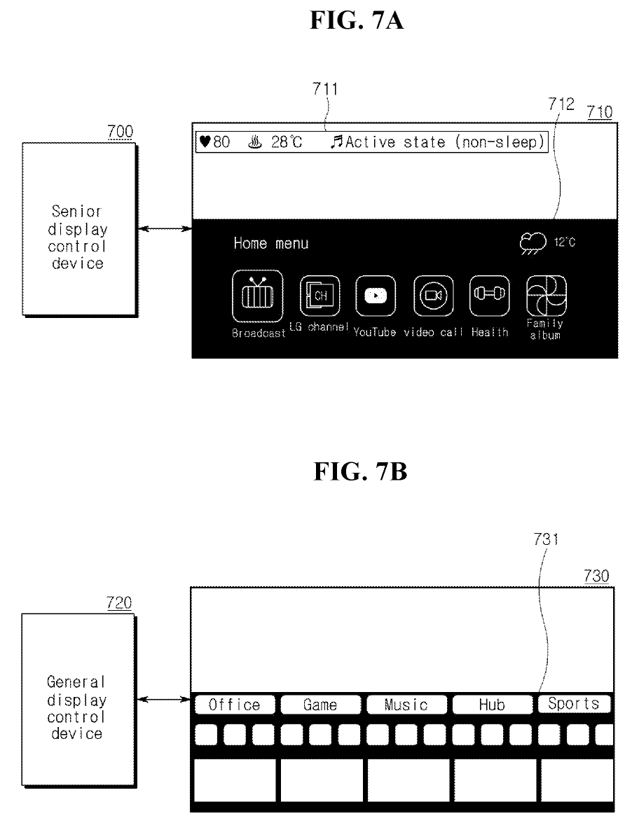

FIGS. 7A and 7B are diagrams showing an operation of varying a user interface displayed on a screen of a display device based on a type of a display control device.

FIG. 7A shows that the display device is connected to a senior display control device, and FIG. 7B shows that the display device is connected to a general display control device.

Referring to FIGS. 7A and 7B, the display control device may be categorized into a senior display control device 700 for senior users and a general display control device 720 for general users, but the type of the display control device is not limited thereto. According to another embodiment, there may be an infant display control device for infant users and a disabled display control device for users with disabilities. The type of the display control device may be distinguished based on a hardware module such as a dip switch or by a parameter value preset by a soft module.

Referring to FIG. 7A, if the display control device is a senior display control device 700, the display control device 700 may control the display device 710 to display a user interface for senior users on the screen of the display device 710. For example, the user interface for senior users may have a larger font size used in the home menu 712 than a general user interface, and the size of the image representing the application may also be larger than the general user interface. Additionally, the user interface for senior users may display biometric information of the wearable device user.

Referring to FIG. 7B, if the display control device is a general display control device 720, the display control device 720 may control the display device 730 to display a general user interface on the screen of the display device 730. For example, the user interface for general users may have a smaller font size used in the home menu 731 than the senior user interface, and the size of the image representing the application may also be smaller than the senior user interface. Additionally, the configuration of the home menu 731 or the configuration of the screen may also differ from the senior user interface.

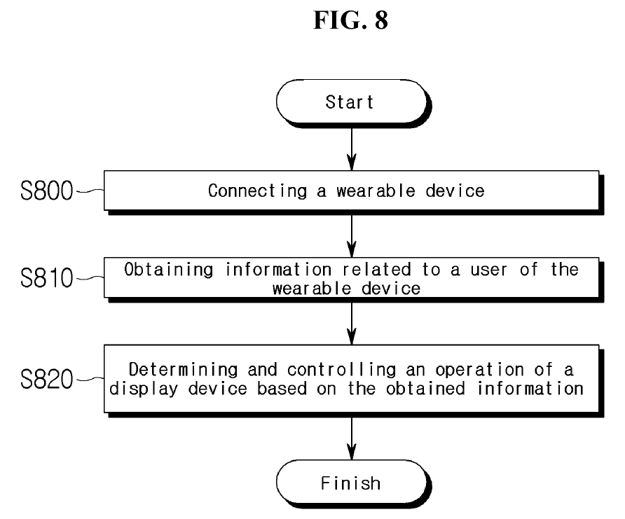

FIG. 8 is a flow chart showing an operation method of a display device according to one embodiment.

FIG. 8 shows the operation of a display device having a configuration directly connecting the display device to the wearable device 140.

Referring to FIG. 8, the operation method of the display device 100 may include an operation of connecting the wearable device S800; an operation of obtaining the information related to the wearable device user S810; and an operation of controlling the operation of the display device based on the obtained information related to the display device S820.

In the operation S800, the display device 100 may be communicatively connected to the wearable device 140 tagged in a preset method (e.g., NFC tagging). In addition, the display device 100 may transmit or receive setting data for Bluetooth communication or Wi-Fi communication through communication with the tagged wearable device 140, and additionally connect Bluetooth or Wi-Fi communication.

In the operation S810, the display device 100 may obtain the information related to the wearable device user transmitted by the wearable device at the preset time interval (e.g., 10ms). According to one embodiment, the wearable device 140 may transmit the information related to the wearable device user to the display device 100 at the preset time interval. According to one embodiment, the information transmitted by the wearable device 140 to the display device 100 may be preset.

In the operation S820, the display device 100 may determine information to be displayed on the screen based on the information related to the wearable device user transmitted by the wearable device 140. Here, the information to be displayed on the screen may include heat rate information, sleep information of the wearable device user, and further include home menu information, indoor temperature information, etc., but the information to be displayed on the screen is not limited thereto.

In the operation S820, as an additional operation, the display device may count the number of packets transmitted by the wearable device 140 at a preset time interval (e.g., 10 seconds).

If the counted number of packets is less than a preset value (e.g., 500 packets), the display device 100 may determine that the wearable device 140 is beyond a valid distance of the display device 100, and may determine not to display the information related to the wearable device user transmitted by the wearable device 140 on the screen.

If the counted number of packets is a preset value or more (e.g., 500 packets), the display device 100 may determine to display the temperature of the wearable device, the heat rate of the wearable device user or whether the wearable device user is sleeping on the screen based on the information related to the wearable device user transmitted by the wearable device 140.

Hence, the display device 100 may display the determined information on the screen.

In the operation S820, as an additional operation, the display device 100 may determine whether the wearable device user is sleeping based on the information related to the wearable device user transmitted by the wearable device 140.

According to one embodiment, if heat rate information is included in the information related to the wearable device user transmitted by the wearable device 140, the display device 100 may determine whether the wearable device user is sleeping based on the heat rate information. For example, the display device 100 may determine that the wearable device user is in a sleep state, if the obtained heat rate is a preset number of times or less.

According to another embodiment, when the information about whether the wearable device user is sleeping is included in the information related to the wearable device user transmitted by the wearable device 140, the display device 100 may determine whether the wearable device user is sleeping based on the corresponding sleep information.

The display device 100 may determine the operation of the display device based on whether the wearable device user is sleeping. According to one embodiment, the operation of the display device may be turning the display device on/off or activating/deactivating the always-on display, but the operation of the display device based on whether the wearable device user is in a sleep state is not limited thereto.

In the operation S820, as an additional operation, the display device 100 may determine whether the wearable device user performs a preset action based on the information related to the wearable device user, and perform an operation corresponding thereto.

According to one embodiment, when receiving trigger information in the packets received from the wearable device 140, the display device 100 may determine that the wearable device user has performed the action in response to the corresponding trigger information.

Once determining that the wearable device user has performed the specific action through the received trigger information, the display device 100 may perform a preset operation corresponding to the action. For example, when receiving trigger information corresponding to a double-pinch action, the display device 100 may determine that the wearable device user has performed a double-pinch, and may output a preset alarm through the speaker. According to one embodiment, in response to the wearable device user’s action, the preset operation of the display device 100 may include turning the display device on/ff, turning the screen on/off, in addition to the alarm output.

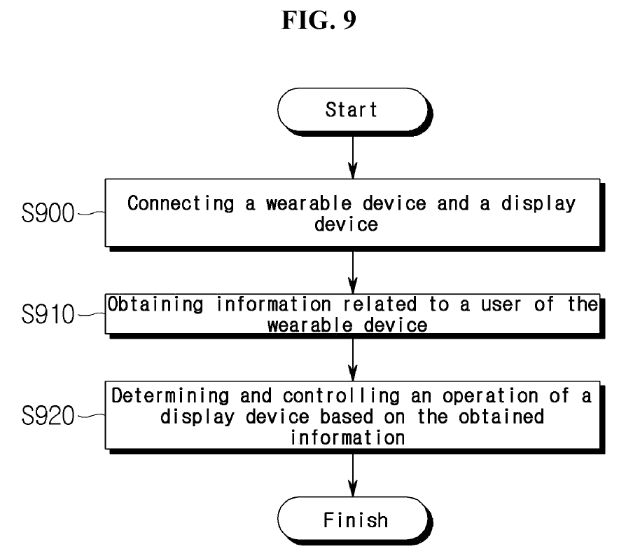

FIG. 9 is a flow chart showing an operation method of a display control device according to another embodiment.

FIG. 9 shows an operation of the display control device 200 provided separately from the display device 250 and configured to control the display device 250, in the configuration in which the display control device is connected to the wearable device 240. According to one embodiment, the display control device 200 may be a device having a conventionally used remote controller as an additional function. Accordingly, the display control device 200 may be configured to control the display device 250 via Bluetooth communication and/or infrared communication.

Referring to FIG. 9, the operation method of the display control device 200 may include an operation S900 of connecting the wearable device 240 and the display device 250; an operation S810 of obtaining the information related to the wearable device user from the wearable device 240; and an operation S920 of determining and controlling the operation of the display device based on the obtained information related to the wearable device user.

In the operation S900, the display control device 200 may be configured to connect a wearable device 240 tagged by a preset method (e.g., NFC tagging). Then, the display device 100 may transmit or receive setting data for Bluetooth communication or Wi-Fi communication through communication with the tagged wearable device 240, and additionally connect Bluetooth or Wi-Fi communication

In the operation S900, the display control device 200 may be communicatively connected to the display device 250. The display control device 200 may control the display device via infrared communication in the same method as a conventional television remote controller controlling a television, and may also be communicatively connected to the display device 250 via Bluetooth communication method.

The display control device 200 may simultaneously perform a Bluetooth master function and a Bluetooth slave function to be communicatively connected to both the display device 250 and the wearable device 240 by using Bluetooth communication method. According to one embodiment, the display control device 200 may operate as a Bluetooth master to be communicatively connected to the wearable device and may operate as a Bluetooth slave to be communicatively connected to the display device 250.

In the operation S910, the display control device 200 may obtain the information related to the wearable device user at a preset time interval (e.g., 10ms) transmitted by the wearable device 240. According to one embodiment, the wearable device 240 may transmit the information related to the wearable device user to the display control device 200 at a preset time interval. According to one embodiment, the information transmitted to the display control device 200 by the wearable device 240 may be preset.

In the operation S920, the display control device 200 may determine information, which will be displayed on the screen of the display device 250, based on the information related to the wearable device user transmitted by the wearable device 240. Here, the information to be displayed on the screen of the display device 250 may include biometric information of the wearable device user such as heat rate information of the wearable device user, whether the wearable device user is sleeping, etc.), and additionally home menu information, indoor information, etc., but the information to be displayed on the screen of the display device 250 is not limited thereto.

In the operation S920, as an additional operation, the display control device 200 may count the number of packets transmitted by the wearable device 240 at a preset time interval (e.g., 10 seconds).

If the counted number of packets is less than a preset value (e.g., 500 packets), the display device 200 may determine that the wearable device 240 is beyond a valid distance of the display device 200, and may determine not to display the information related to the wearable device user transmitted by the wearable device 240 on the screen.

If the counted number of packets is a preset value or more (e.g., 500 packets), the display device 200 may determine to display the temperature of the wearable device, the heat rate of the wearable device user or whether the wearable device user is sleeping on the screen based on the information related to the wearable device user transmitted by the wearable device 240.

Hence, the display control device 200 may transmit information to the display device so that the determined information can be displayed on the screen of the display device 250.

In the operation S920, as an additional operation, the display control device 200 may determine whether the wearable device user is sleeping based on the information related to the wearable device user transmitted by the wearable device 240.

According to one embodiment, when heat rate information is included in the information related to the wearable device user transmitted by the wearable device 240, the display control device 200 may determine whether the wearable device user is sleeping based on the heat rate information. For example, the display control device 200 may determined that the wearable device user is in a sleep state, if the obtained heat rate is a preset number of times.

According to another embodiment, when sleep information is included in the information related to the wearable device user transmitted by the wearable device 240, the display control device 200 may determine whether the wearable device user is sleeping based on the corresponding sleep information.

The display control device 200 may determine the operation of the display device 250 based on whether the wearable device user is sleeping. According to one embodiment, the operation of the display device may be turning the display device on/off, turning the screen on/off, or activating/deactivating the always-on display, but the operation of the display device based on the sleep information is not limited thereto. According to one embodiment, the display control device 200 may be configured to control the operation of the display device 250 by using a remote controller function provided therein.

In the operation S920, as an additional operation, the display control device 200 may control the display device 250 to display different user interfaces on the screen of the display device 250 based on the type of the display control device 250.

According to one embodiment, the display control device 200 be configured to set the operation of the display control device 200, using a hardware method (e.g., a dip switch) or a software method (e.g., setting operation parameters). According to one embodiment, the display control device 200 may include a senior display control device, a normal display control device, a disabled display control device, and an infant display control device.

According to one embodiment, the display control device 200 may control the display device 250 to display a user interface corresponding to the type of the display control device 200 on the screen of the display device 250.

The display device 250 may include a user interface having different settings corresponding to the type of the display control device 250. For example, a user interface for a senior display control device may be set to use a larger font than another user interface. The display control device 200 control the display device 250 to display the user interface corresponding to its type on the screen of the display device 250.

In the above, it is described that all components constituting the embodiments are connected as one or connected to operate, but it is not limited to the embodiments. That is, within the scope of the embodiments, all of the components are selectively coupled as at least one to operate.

Furthermore, each of all the components may be implemented as one single independent hardware, some or all of the components may be selectively combined and implemented as a computer program having program modules that perform some or all of the functions of the combined hardware in one or more modules. The codes and code segments provided in the computer program may be easily derived by those skilled in the art to which the present disclosure pertains.

Such a computer program may be stored in a computer-readable storage medium and read and executed by a computer, thereby implementing the embodiments of the present disclosure. The storage medium of the computer program may include a magnetic recording medium, an optical recording medium, etc.

The described embodiments are intended to include all alternatives, modifications and variations that fall within the true spirit and scope of the appended claims. Furthermore, to the extent that the term "comprises", “include” or “has” is used in the detailed description or claims, such term is intended to be included in a similar manner as "configured of" as the term "consisting of" is to be interpreted when used as a transitional word in the claims.

Terminology that is used in the present disclosure is limited to only for embodiments herewith but made only to make it easy to understand the present disclosure. Terms of respective elements used in the following description are terms defined taking into consideration of the functions obtained in the present invention. Therefore, these terms do not limit technical elements in the present invention. Further, the defined terms of the respective elements will be called other terms in the art.

The methods disclosed in the present disclosure may comprises one or more operations or steps. The operations and/or steps of the method may be interchanged with each other without departing from the scope of the claims. In other words, unless a specific order for the operations or steps is specified, the order and/or use of particular operations and/or steps may be modified without departing from the scope of the claims.

As used herein, the phrase “at least one of” the list of items may refer to any combination of those items, including single members. For example, “at least one of a, b, or c” is intended to include a, b, c, a-b, a-c, b-c, and a-b-c, as well as any combination of multiples of the same element (e.g., a-a, a-a-a, a-a-b, a-a-c, a-b-b, a-c-c, b-b, b-b-b, b-b-c, c-c, and c-c-c or any other ordered sequence of a, b, and c).

As used herein, the term “determining” encompasses a wide variety of operations. For example, “determining” may include calculating, computing, processing, deriving, investigating, looking up (e.g., looking up in a table, database, or other data structure), ascertaining, etc. Additionally, “determining” may include receiving (e.g., receiving information), accessing (e.g., accessing data in a memory), etc. additionally, “determining” may include resolving, selecting, choosing, establishing, etc.

The above description is merely an example of the technical idea of the present disclosure, and those skilled in the art will appreciate that various modifications and variations can be made without departing from the essential characteristics of the present disclosure.

Although the present invention has been described with reference to the exemplified drawings, it is to be understood that the present invention is not limited to the embodiments and drawings disclosed in this specification, and those skilled in the art will appreciate that various modifications are possible without departing from the scope and spirit of the present invention. Further, although the operating effects according to the configuration of the present invention are not explicitly described while describing an embodiment of the present invention, it should be appreciated that predictable effects are also to be recognized by the configuration.

1. A display device comprising:
an input/output interface communicatively connected to a wearable device and comprising a screen; and
at least one processor,
wherein the at least one processor is configured to:
obtain information related to a wearable device user transmitted by the wearable device; and
control an operation of the display device based on the obtained information related to the wearable device user.

2. The display device of claim 1, wherein the information related to the wearable device user comprises at least one of biometric information of the wearable device user, location information of the wearable device user and activity information of the wearable device user.

3. The display device of claim 1, wherein the at least one processor is configured to:
determine information, which will be displayed on the screen, based on the information related to the wearable device user; and
display the determined information on the screen.

4. The display device of claim 1, the at least one processor is configured to:
count a number of packets transmitted by the wearable device at a preset time interval;
determine whether the number of packets is less than a preset value;
determine not to display the information related to the wearable device user on the screen, when the number of packets is less than the preset value.

5. The display device of claim 1, wherein the at least one processor is configured to:
determine whether the wearable device user is sleeping based on the information related to the wearable device user; and
turn the display device off or turn the screen off, when determining that the wearable device user is sleeping.

6. The display device of claim 1, wherein the at least one processor is configured to:
obtain trigger information transmitted by the wearable device; and
perform a preset operation of the display device in response to the trigger information, and
wherein the trigger information is transmitted by the wearable device when the wearable device user performs a preset action.

7. A display control device comprising:
an input/output device communicatively connected to a wearable device and a display device; and
at least one processor configured to:
obtain information related to a wearable device user transmitted by the wearable device;
determine an operation of the display device based on the obtained information related to the wearable device user; and
control the display device based on the determined operation.

8. The display control device of claim 7, wherein the information related to the wearable device user comprises at least one of biometric information of the wearable device user, location information of the wearable device user and activity information of the wearable device user.

9. The display control device of claim 7, wherein the at least one processor is configured to:
determine first information, which will be displayed on a screen of the display device, based on the information related to the wearable device user;
transmit the determined first information to the display device; and
control the display device to display the first information on the screen.

10. The display control device of claim 7, wherein the at least one processor is configured to:
count the number of packets transmitted by the wearable device at a preset time interval;
determine whether the number of packets is less than a preset value;
determine not to display the information related to the wearable device user on the screen, when the number of packets is less than the preset value.

11. The display control device of claim 7, wherein the at least one processor is configured to:
determine whether the wearable device user is sleeping based on the information related to the wearable device user; and
control the display device to be turned off or to turn the screen off, when determining that the wearable device user is sleeping.

12. The display control device of claim 7, wherein the at least one processor is configured to:
obtain trigger information transmitted by the wearable device; and
control the display device to perform a preset operation of the display device in response to the trigger information, and
wherein the trigger information is transmitted by the wearable device when the wearable device user performs a preset action.

13. The display control device of claim 7, further comprising:
a module configured to set the type of the display control device,
wherein the at least one processor is configured to:
determine a user interface corresponding to the type of the display control device set by the module; and
control the display device to display the user interface on the screen of the display device.

14. The display control device of claim 7, wherein the input/output interface comprises two Bluetooth communication modules,
wherein one of the two Bluetooth communication modules operates as a master to be communicatively connected to the wearable device, and
wherein the other one of the two Bluetooth communication modules operates as a slave to be communicatively connected to the display device.

15. An operation method of a display device comprising:
communicatively connecting to a wearable device;
obtaining information related to a wearable device user transmitted by the wearable device;
controlling an operation of the display device based on the obtained information related to the wearable device user.

16. An operation method of a display control device comprising:
connecting to a wearable device;
connecting to a display device;
obtaining information related to a wearable device user transmitted by the wearable device;
determining an operation of the display device based on the obtained information related to the wearable device user; and
controlling the display device based on the determining operation.
